(19) United States
(12) Patent Application Publication
(10) Pub. No.: US 2026/0134959 A1
(43) Pub. Date: May 14, 2026

(54) MEDICAL DEVICE WITH ELECTRONIC MEDICAL RECORD INTEGRATION

(71) Applicant: Welch Allyn, Inc., Skaneateles Falls, NY (US)

(72) Inventor: Thomas Henry Briggs, Frederick, MD (US)

(21) Appl. No.: 19/388,694

(22) Filed: Nov. 13, 2025

Related U.S. Application Data
(60) Provisional application No. 63/720,313, filed on Nov. 14, 2024.

(51) Int. Cl. G16H 10/60; G16H 40/67

(52) U.S. Cl. CPC G16H 10/60; G16H 40/67

(57) ABSTRACT

A medical device includes one or more modules configurable to connect to one or more sensors for capturing one or more physiological variable measurements and a display screen for displaying the physiological variable measurements. The medical device receives data identifying a patient, retrieves an electronic medical record of the patient acquired from a system responsible for maintaining the electronic medical record, and stores the electronic medical record on the at least one memory device. The medical device determines an interruption of a connection with the system responsible for maintaining the electronic medical record. The medical device updates the electronic medical record to include the physiological variable measurements captured by the one or more sensors while the interruption persists.

### CROSS-REFERENCE TO RELATED APPLICATION(S)

This application claims the benefit of U.S. Provisional Application No. 63/720,313, filed Nov. 14, 2024, the disclosure of which is hereby incorporated by reference in its entirety.

### BACKGROUND

Electronic Medical Records (EMRs) are digital versions of patient charts and medical histories. EMRs are used by healthcare providers to store and manage patient information, including demographics, medical history, medications, treatment plans, and test results. EMRs enhance the efficiency of healthcare delivery by allowing for easy access and sharing of information among healthcare providers, which can improve coordination and reduce errors.

Key benefits of EMRs can include improved patient care by enabling quick access to comprehensive patient data, which supports better clinical decision-making and enhances patient safety. EMRs also provide increased efficiency by streamlining workflows, reducing paperwork, and minimizing duplication of tests, which saves time for both healthcare providers and patients. EMRs can also improve data analytics for population health management and research, and provide valuable insights into health trends and outcomes. Further, EMRs improve patient engagement by offering patient portals that allow individuals to access their health information, schedule appointments, and communicate with healthcare providers.

However, healthcare facilities which depend on EMRs often revert back to paper records due to outages of EMR systems, which have occurred with increasing frequency. EMR systems have proven to be fragile in view of troublesome software updates, environmental disasters, communications and power infrastructure interruptions, and even wars which can wreak havoc on existing clinical practices and create opportunities for clinical errors.

### SUMMARY

In general terms, the present disclosure relates to a medical device for monitoring a patient. In one possible configuration, the medical device stores an electronic medical record of the patient that is maintained by another system, and the medical device updates the electronic medical record when a connection with the other system is interrupted. Various aspects are described in this disclosure, which include, but are not limited to, the following aspects.

One aspect relates to a medical device, comprising: one or more modules configurable to connect to one or more sensors for capturing one or more physiological variable measurements; a display screen for displaying the physiological variable measurements; at least one processing device; and at least one memory device storing software instructions that, when executed by the at least one processing device, cause the at least one processing device to: receive data identifying a patient; retrieve an electronic medical record of the patient acquired from a system responsible for maintaining the electronic medical record; store the electronic medical record on the at least one memory device; determine an interruption of a connection with the system responsible for maintaining the electronic medical record; and update the electronic medical record to include the physiological variable measurements captured by the one or more sensors while the interruption persists.

Another aspect relates to a method of monitoring a patient, the method comprising: receiving data identifying a patient; retrieving an electronic medical record of the patient acquired from a system responsible for maintaining the electronic medical record; storing the electronic medical record on at least one memory device; receiving one or more physiological variable measurements from one or more sensors; determining an interruption of a connection with the system responsible for maintaining the electronic medical record; and updating the electronic medical record to include the physiological variable measurements captured by the one or more sensors while the interruption persists.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a front isometric view of a medical device that can be used to monitor one or more physiological variables of a patient.

FIG. 2 is a rear isometric view of the medical device of FIG. 1.

FIG. 3 is a front view of the medical device of FIG. 1.

FIG. 4 is a bottom view of the medical device of FIG. 1.

FIG. 5 schematically illustrates an example of the medical device of FIG. 1.

FIG. 6 schematically illustrates an example of a method of monitoring a patient that can be performed by the medical device of FIG. 1.

FIG. 7 illustrates an example of a graphical user interface displayed on a display screen of the medical device of FIG. 1.

FIG. 8 illustrates another example of a graphical user interface displayed on the display screen of the medical device of FIG. 1, the graphical user interface is displayed in response to a selection of an EMR tab in the graphical user interface of FIG. 7.

### DETAILED DESCRIPTION

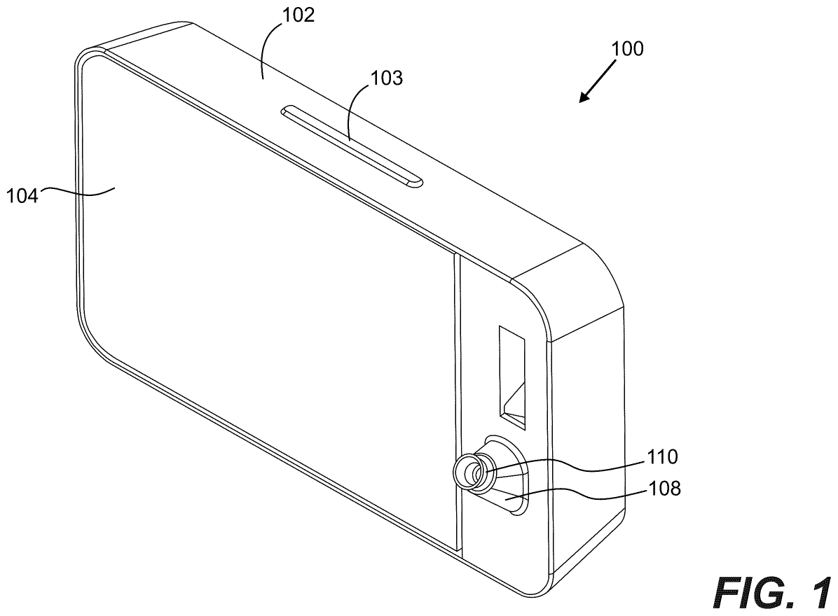

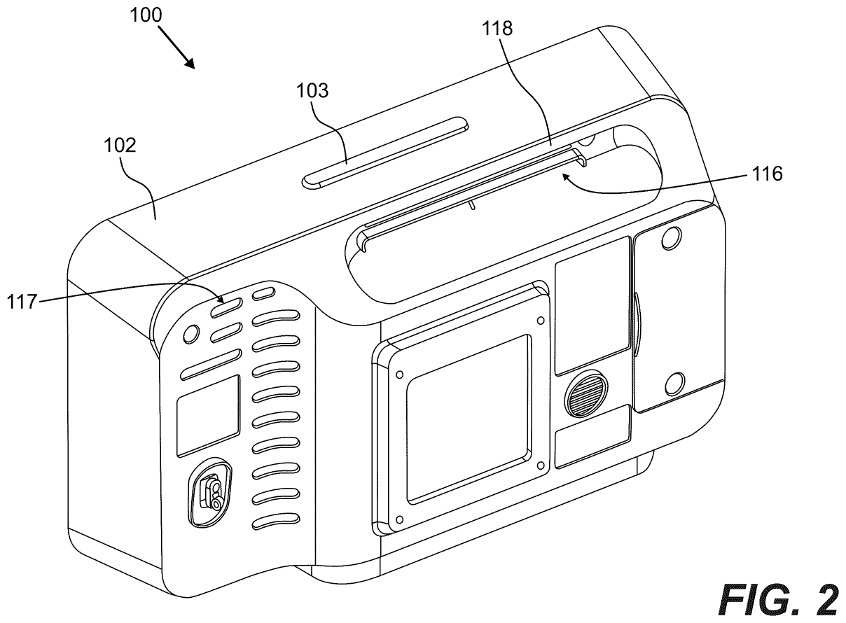

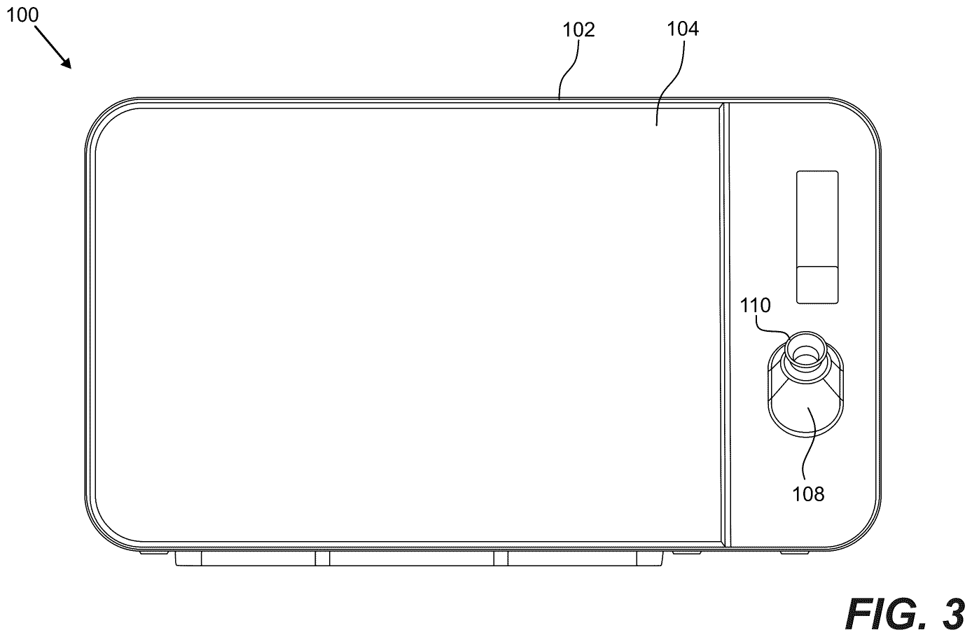

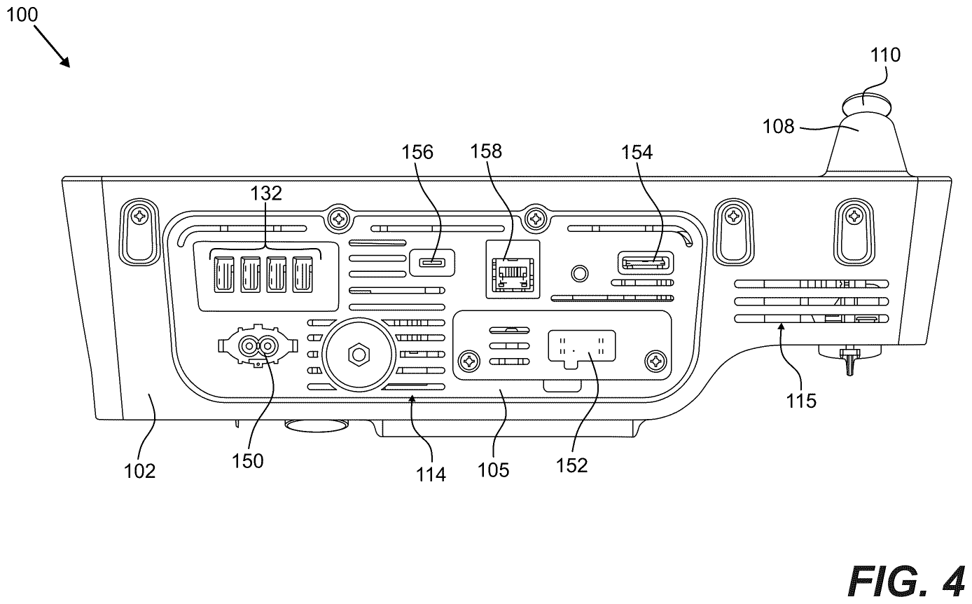

FIG. 1 is a front isometric view of a medical device 100 that can be used to monitor one or more physiological variables of a patient. FIG. 2 is a rear isometric view of the medical device 100. FIG. 3 is a front view of the medical device 100. FIG. 4 is a bottom view of the medical device 100. As shown in FIGS. 1-4, the medical device 100 includes a housing 102, and a display screen 104 mounted on a front portion of the housing 102. The display screen 104 is a touch sensitive touchscreen that receives inputs from a user of the medical device 100 such as a caregiver, a physician, a clinician, a nurse, or other trained healthcare professional.

The medical device 100 is designed for use in a clinical setting. For example, the medical device 100 can be used to continuously monitor one or more physiological variables of a patient admitted to a medical facility such as a hospital, medical clinic, and the like. Additionally, the medical device 100 can operate as a spot monitor for episodic monitoring of one or more physiological variables of a plurality of patients in the medical facility.

The medical device 100 includes one or more modules configurable to connect to one or more sensors that can be used to measure the one or more physiological variables including, without limitation, blood pressure, blood oxygen saturation (SpO2), heart rate, pulse rate, body temperature, and respiration rate. The medical device 100 can include a thermometer module 108 to measure body temperature, a module configured to connect to a non-invasive blood pressure cuff to measure systolic and diastolic blood pressure, a module configured to connect to a pulse oximeter to measure blood oxygen saturation and pulse rate, and can include additional types of modules and sensors for capturing additional types of physiological variable measurements.

The sensors can include cables that terminate into connectors that plug into various ports of the modules included on the housing 102 of the medical device 100 for transmission of data to the medical device 100. As shown in FIG. 4, the housing 102 includes a first port 150 for connecting the non-invasive blood pressure cuff and a second port 152 for connecting the pulse oximeter. Optionally, the medical device 100 can include a third port 154 for connecting the medical device 100 to a printer. Also, the medical device 100 can include one or more universal serial bus (USB) ports 132, a USB-C port 156, and a registered jack (RJ) interface 158. In some examples, at least some of the sensors 112 wirelessly transmit data to the medical device 100 via a wireless connection established through Wi-Fi, Bluetooth, or another wireless protocol.

In further examples, the housing 102 includes a port for connection of a scanner to the medical device 100. The scanner can be used to scan machine-readable data such as a barcode including linear barcodes, quick-response (QR) codes, and the like that identify a person such as a patient who is being monitored by the medical device 100 or a user of the medical device 100 (e.g., a nurse, a doctor, a caregiver, and the like). The machine-readable data can further identify an object such as medical equipment or a medication being administered to the patient.

In the example shown in FIGS. 1-4, the thermometer module 108 is integrated with the medical device 100. The thermometer module 108 includes a port 110 for housing a handheld probe that can be orally inserted into the mouth of a patient to take a temperature reading. Alternatively, the housing 102 of the medical device 100 can be configured to house a handheld probe that can be inserted into an ear of the patient to take a temperature reading.

The medical device 100 receives data from the sensors 112 for processing. The medical device 100 generates and displays numerical values and/or waveforms based on the data received from the sensors 112 for monitoring the one or more physiological variables of the patient. The medical device 100 displays the numerical values and/or waveforms of the one or more physiological variables on the display screen 104. Also, the medical device 100 generates alarms when the data received from the sensors 112 indicates an alarm condition is detected.

The alarms generated by the medical device 100 can include a visual alarm displayed on the display screen 104 and an audible alarm emitted by a speaker of the medical device 100. An additional visual alarm can be displayed on an illumination unit 103, which is positioned on the top portion of the housing 102 of the medical device 100. The illumination unit 103 allows for distant viewing of critical alarms. The critical alarms generated on the illumination unit 103 can be viewed across a 360 degree field of view around the medical device 100. In some examples, both a visual alarm is displayed on the display screen 104 and a further visual alarm is generated on the illumination unit 103 to indicate a critical status of a detected alarm condition.

The illumination unit 103 can include one or more light-emitting diodes (LEDs) that emit light that is visible through a translucent cover. As an illustrative example, the illumination unit 103 can emit light having a color (e.g., yellow or red) based on a severity of the alarm. Additionally, or alternatively, the illumination unit 103 can emit light flashes based on a predetermined pattern associated with the severity of the alarm (e.g., an increased blinking rate to indicate severe conditions and a decreased blinking rate to indicate less severe conditions).

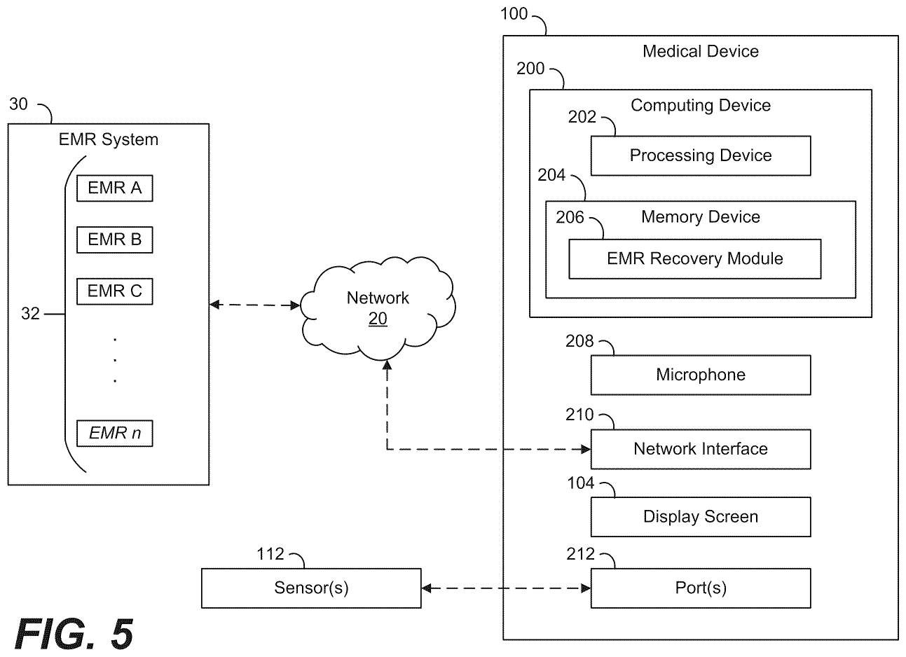

FIG. 5 schematically illustrates an example of the medical device 100. As shown in FIG. 5, the medical device 100 includes a computing device 200 having at least one processing device 202 and at least one memory device 204 that stores software instructions that, when executed by the at least one processing device 202, cause the at least one processing device 202 to perform the various aspects, functions, and operations described herein.

The at least one processing device 202 is an example of a processing unit such as a central processing unit (CPU). The at least one processing device 202 can include one or more CPUs. In some examples, the at least one processing device 202 includes one or more digital signal processors, field-programmable gate arrays, and/or other types of electronic circuits.

The at least one memory device 204 is an example of a computer-readable data storage device that operates to store data and instructions for execution by the at least one processing device 202. The at least one memory device 204 includes computer-readable media, which includes any media that can be accessed by the at least one processing device 202. The computer-readable media can include computer-readable storage media and computer-readable communication media. The computer-readable storage media includes volatile and nonvolatile, removable and non-removable media implemented in any device that can store information such as computer-readable instructions, data structures, program modules, or other data. The computer-readable storage media can include random access memory, read only memory, electrically erasable programmable read only memory, flash memory, and other memory technology, including any medium that can be used to store information that can be accessed by the at least one processing device 202. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. The computer-readable communication media can include wired media such as a wired network or direct-wired connection, and wireless media such as acoustic, radio frequency, infrared, and other wireless media. Combinations of any of the above are within the scope of computer-readable media.

As shown in FIG. 5, the at least one memory device 204 stores an electronic medical record (EMR) recovery module 206 which caches an electronic medical record (EMR) of a patient who is being monitored by the medical device 100 for storage on the at least one memory device 204 of the medical device. The EMR of the patient is temporarily stored on the at least one memory device 204 such that it is purged or otherwise deleted from the at least one memory device 204 based on one or more conditions being satisfied such as an expiration of a time period, a quantity of EMRs cached on the at least one memory device 204, and/or the patient's discharge from the medical facility where the medical device 100 is located.

In some examples, the medical device 100 includes a microphone 208 that can be used to capture audio from a user of the medical device 100. As will be described further below, in some examples the computing device 200 is configured to process the audio to transcribe voice notes from the user of the medical device 100. The voice notes can include clinically relevant observations of the patient that are made by the user of the medical device 100.

The medical device 100 includes a network interface 210 that facilitates connection to the network 20 that can include any type of wired or wireless connections or any combinations thereof. The network interface 210 can include wired interfaces and/or wireless interfaces. For example, the network interface 210 can be used to wirelessly connect the medical device 100 to the network 20 such as through Wi-Fi and the like. Alternatively, or additionally, the network interface 210 can be used to connect the medical device 100 to the network 20 using wired connections such as through Ethernet or Universal Serial Bus (USB) cables.

As further shown in FIG. 5, the one or more sensors 112 can plug into one or more ports 212 of the medical device 100 (e.g., the first port 150, the second port 152, etc.), and/or that can wirelessly transmit data to the medical device 100, and/or that can be integrated with the medical device 100 in accordance with the examples described above.

The medical device 100 also communicates with an electronic medical record (EMR) system 30 via the network 20. The EMR system 30 maintains a plurality of EMRs 32 for a plurality of patients. The EMR system 30 can be operated by a healthcare service provider such as a hospital or medical clinic to maintain the plurality of EMRs 32 for the plurality of patients.

The physiological measurements captured by the one or more sensors 112 are sent to the EMR system 30 via the network 20 for storage in the EMR 32 of a patient who is being monitored by the medical device 100. Additionally, the medical device 100 can cache the EMRs 32 from the EMR system 30 for temporary storage on the at least one memory device 204.

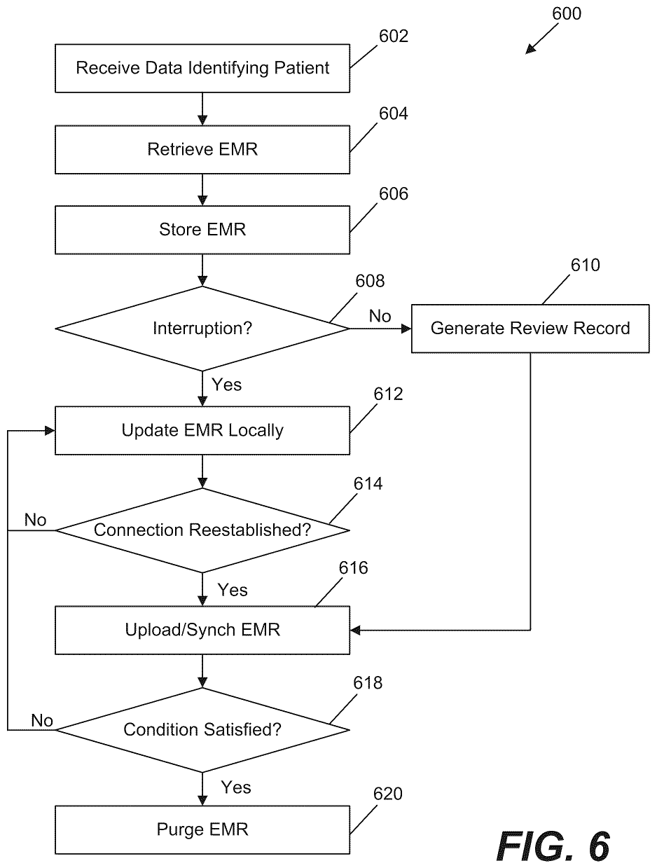

FIG. 6 schematically illustrates an example of a method 600 of monitoring a patient that can be performed by the medical device 100. The method 600 can be performed by the EMR recovery module 206 stored on the at least one memory device 204 of the medical device 100.

The method 600 can improve patient safety when the connection between the medical device 100 and the EMR system 30 is interrupted by ensuring that clinically relevant information in the EMR 32 of a patient remains available on the medical device 100. Further, the method 600 can ensure the EMR of the patient remains accurate and up-to-date even when the connection between the medical device 100 and the EMR system 30 is interrupted.

As shown in FIG. 6, the method 600 includes an operation 602 of receiving data identifying a patient. In some examples, operation 602 includes receiving the data identifying the patient via a scan of a machine-readable data on an item worn by the patient (e.g., wrist bracelet) or on an item associated with the patient such as a patient hospital bed and the like. As described above, a scanner of the medical device 100 can be used to scan machine-readable data that identifies the patient who is being monitored by the medical device 100.

In further examples, the operation 602 includes receiving the data identifying the patient via an entry received on the display screen 104. For example, once the patient is admitted to the healthcare facility, the patient can be selected from a menu that includes a list of patients admitted to the healthcare facility. Alternatively, the patient's name or the patient's ID number can be manually typed on the display screen 104 by a user of the medical device 100.

The method 600 includes an operation 604 of retrieving the EMR of the patient acquired from the EMR system 30. As described above, the EMR system 30 is responsible for maintaining the EMR of the patient. Operation 604 can include retrieving the EMR of the patient from the EMR system 30 over the network 20 (see FIG. 5).

The method 600 includes an operation 606 of storing the EMR 32 of the patient on the at least one memory device 204 of the medical device 100. In some examples, operation 606 includes caching the EMR 32 onto the at least one memory device 204. In some examples, an entirety of the EMR 32 is stored on the at least one memory device 204. In other examples, a portion of the EMR 32 is stored on the at least one memory device 204. In some examples, the storage of the EMR 32 on the at least one memory device 204 is configurable to store one or more of physiological variable measurements, physical assessments, notes and history, diagnoses, test results, medication records, and diagnostic imaging obtained from other devices.

The method 600 includes an operation 608 of determining whether there is an interruption of a connection with the EMR system 30. When the connection is not interrupted (i.e., “No” in operation 608), the method 600 proceeds to an operation 610 of generating a review record based on the data captured by the one or more sensors 112 or otherwise entered on the display screen 104 of the medical device 100. The review record is a local copy of patient assessment maintained by the medical device 100 that may include limited patient information obtained from the EMR 32 such as patient name, clinician name, and medical record number. The review record is generated by the medical device 100 when not operating under an emergency EMR mode. The review record can later be used to update the EMR 32 of the patient.

When the connection is interrupted (i.e., “Yes” in operation 608), the method 600 proceeds to an operation 612 of updating the EMR 32 locally on the medical device 100. For example, operation 612 can include adding the measurements of the one or more physiological variables captured by the one or more sensors 112 to the EMR 32. Operation 612 can further include adding clinical observations that are received via the display screen 104 of the medical device 100 to the EMR 32. In some further examples, operation 612 includes processing audio of the user captured by the microphone 208 of the medical device 100 to transcribe voice notes from the user of the medical device 100, and storing the voice notes to the EMR 32.

The method 600 includes an operation 614 of determining whether the connection with the EMR system 30 is reestablished. When the interruption of the connection with the EMR system 30 persists such that the connection is not reestablished (i.e., “No” in operation 614), the method 600 returns to operation 612 to continue to update the EMR 32 stored locally on the on the at least one memory device 204 of the medical device 100.

When the connection with the EMR system 30 is reestablished (i.e., “Yes” in operation 614), the method 600 proceeds to an operation 616 of uploading the EMR 32 to the EMR system 30. In operation 616, the EMR 32 includes physiological variable measurements captured by the one or more sensors 112, clinical observations and notes on medication administration received via the display screen 104 or elsewhere, and transcriptions of voice notes captured by the microphone 208 while the connection with the EMR system 30 was disrupted. In this manner, the EMR system 30 receives an updated version of the EMR 32 thereby mitigating data loss that can result from interruption of the connection to the medical device 100.

In some examples, storage of the EMR 32 on the at least one memory device 204 of the medical device 100 is maintained after the EMR 32 is uploaded to the EMR system 30. In some examples, operation 616 includes synchronizing the EMR 32 uploaded to the EMR system 30 with the EMR 32 stored locally on the at least one memory device 204. In this manner, the EMR 32 maintained by the EMR system 30 is continuously synchronized with the EMR 32 stored on the medical device 100 to mitigate data loss due to intermittent disruptions of the connection between the medical device 100 and the EMR system 30.

The method 600 includes an operation 618 of determining whether one or more conditions are satisfied. The one or more conditions are configurable by a healthcare service provider such as a hospital or medical clinic where the medical device 100 is being used for monitoring the patient. The one or more conditions can include at least one of expiration of a predetermined time period, discharge of the patient from the medical facility or clinic, and reaching a capacity of the at least one memory device 204 of the medical device 100.

When the one or more conditions are not satisfied (i.e., “No” in operation 618), the method 600 returns to operation 612 to continue to update the EMR 32 stored locally on the on the at least one memory device 204 of the medical device 100. Thus, the EMR 32 is maintained locally on the medical device 100 so long as the one or more conditions are not satisfied.

When the one or more conditions are satisfied (i.e., “Yes” in operation 618), the method 600 proceeds to an operation 620 of purging the EMR 32 from the at least one memory device 204 of the medical device 100. In this manner, the method 600 mitigates the potential for unintentional disclosure or loss of protected health information (PHI), which is any information that can identify the patient and relates to their health, including past, present, or future health.

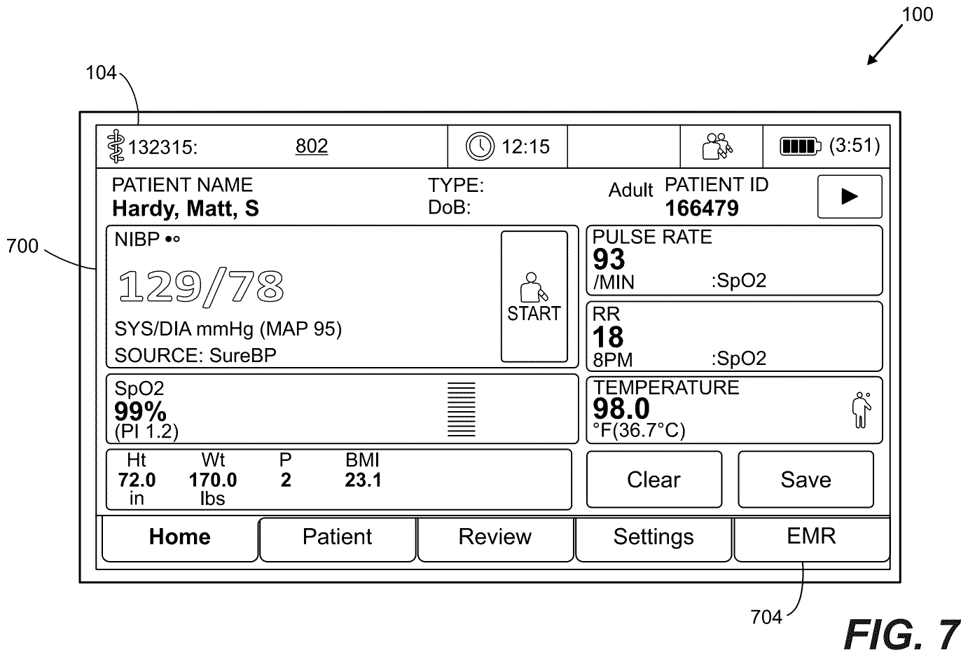

FIG. 7 illustrates an example of a graphical user interface 700 displayed on the display screen 104 of the medical device 100. In this example, the graphical user interface 700 includes a monitoring area 702 that displays measurements of one or more physiological variables captured by the one or more sensors 112 such as non-invasive blood pressure (systolic and diastolic), blood oxygen saturation (SpO2), pulse rate, respiration rate, body temperature, and other data such as patient demographic data (e.g., patient name, date of birth, patient ID number), and other measurements such as the height, weight, and body mass index (BMI).

The graphical user interface 700 further includes an EMR tab 704, which is a shortcut on the EMR 32 stored locally on the medical device 100. As discussed above, the EMR 32 is continuously updated to include the physiological variable measurements displayed in the monitoring area 702, which are captured by the one or more sensors 112.

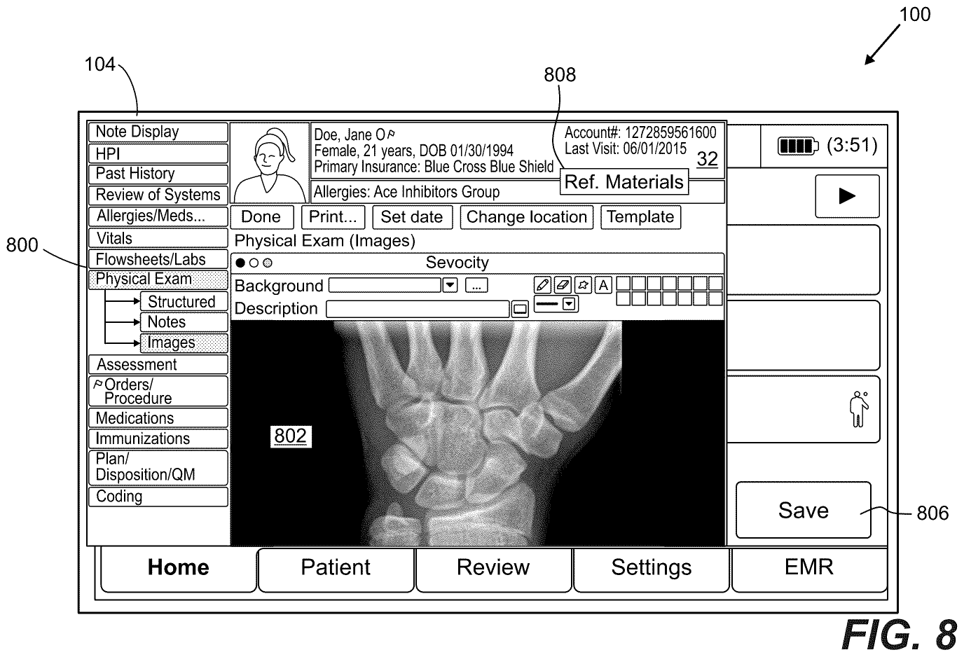

FIG. 8 illustrates another example of a graphical user interface 800 displayed on the display screen 104 of the medical device of 100, the graphical user interface 800 is displayed in response to a selection of the EMR tab 704 in the graphical user interface 700 of FIG. 7. The graphical user interface 800 includes a display of the EMR 32. In this example, the EMR 32 includes a diagnostic image 802 captured by another device such as an X-ray machine, a CT (computed tomography) scan machine, an ultrasound machine, fundus imager, a dermatoscope, and other imaging devices. Accordingly, by storing the EMR 32 locally on the medical device 100, diagnostic imaging can be displayed on the display screen 104 of the medical device 100.

As shown in FIG. 8, the EMR 32 includes a plurality of tabs to display different pieces of information on the display screen 104 of the medical device 100. For example, the EMR 32 can include a note display tab, a history of present illness (HPI) tab, a past health history tab, a review of systems tab, an allergies and medications tab, a vital signs tab, a flowsheets/labs tab, a physical exam tab, an assessment tab, an orders tab, a medications tab, an immunizations tab, a plan/disposition tab, and other tabs for displaying additional information.

By storing the EMR 32 locally on the medical device 100, error checking for drug interactions and medication administration errors can be performed by the medical device 100 even when the connection with the EMR system 30 is interrupted. For example, the EMR 32 stores information such as the allergies and current medications being taken by the patient, and this information can be used to determine whether a new medication is compatible with the allergies and current medications of the patient. The error checking for drug interactions and medication administration errors can automatically be performed once a machine-readable code of the new medication is scanned by the scanner connected to the medical device 100.

When the error checking identifies a drug interaction potentially harmful to the patient or a medication administration error, the medical device 100 issues an alarm. For example, the illumination unit 103, which is positioned on the top portion of the housing 102 of the medical device 100, can illuminate to display a visual alarm. Additionally, or alternatively, the visual alarm can be displayed on the can be displayed on the graphical user interface 800 displayed on the display screen 104 of the medical device of 100. In further examples, an audible alarm can be issued by one or more speakers on the medical device 100.

In this manner, storing the EMR 32 locally on the medical device 100 can improve patient safety when connection to the EMR system 30 is disrupted by preventing drug interactions potentially harmful to patients and/or medication administration errors.

As shown in FIG. 8, the graphical user interface 800 includes a save icon 806 that can be selected by a user of the medical device 100 to save the updates to the EMR 32. For example, the save icon 806 can be selected to save to the EMR 32 physiological variable measurements captured by the one or more sensors 112, new notes and assessments including clinical observations and medication administration received via the display screen 104 or elsewhere, and voice notes transcribed from the audio captured by the microphone 208.

The graphical user interface 800 further includes a reference materials tab 808 that is selectable to display on the display screen 104 reference materials and other publications relevant to a condition of the patient determined from the EMR 32. For example, through partnering with publishers, the medical device 100 can offer electronic copies of physician's desk reference, or similar types of reference materials. As an illustrative example, when the HPI tab of the EMR 32 indicates that the patient has sepsis, selection of the reference materials tab 808 causes the display screen 104 to display reference materials and other publications discussing symptoms and causes of sepsis, and other relevant information for treating sepsis.

Referring now to FIGS. 7 and 8, the EMR tab 704 allows a user of the medical device 100 to toggle between the graphical user interface 700 displaying the monitoring area 702 that displays measurements of one or more physiological variables captured by the one or more sensors 112 and the graphical user interface 800 displaying the EMR 32 of the patient.

Given the foregoing, the display of the EMR 32 on the display screen 104 of the medical device 100 is a function separate from the primary functions of the medical device 100, which include capturing measurements of physiological variables and other relevant clinical data of the patient. In view of FIGS. 7 and 8, a user of the medical device 100 such as a clinician can toggle back and forth between the monitoring area 702 and the EMR 32. In some examples, the functionality of displaying the EMR 32 is independent of the primary function of monitoring the patient. Alternatively, the function of displaying the EMR 32 can be integrated into the primary functions of the medical device 100. Further, storing the electronic medical record locally on the medical device 100 allows diagnostic imaging (e.g., X-rays, CT scans, and the like) to be displayed on the display screen 104 of the medical device 100. Also, the medical device 100 can include audio processing to allow clinician voice notes to be collected by the microphone 208 for later transcription by the computing device 200, or by another system or device.

In the event that the EMR system 30 is unavailable, the medical device 100 allows clinicians to view a cached copy of the EMR 32, add new notes and assessments, record medication administration, check medications against prescriptions, view the saved diagnostic images, and/or perform other related functions. While the outage of the EMR system 30 continues, the medical device 100 operates as the patient record. In some examples, the medical device 100 is portable such that it can move with the patient. While the EMR system 30 is unavailable, the medical device 100 acts in lieu of the EMR system 30. When the EMR system 30 is back up and running, the medical device 100 is programmed to synchronize data collected on the medical device 100 while the connection with the EMR system 30 was disrupted.

Advantages of the medical device 100 include maintaining clinical workflows and efficiencies that depend on the EMR system 30 when connection with the EMR system 30 is disrupted. Further, the medical device 100 can perform error checking for dangerous drug interactions, medication administrator errors, and the like even when the connection with the EMR system 30 is disrupted. Such functions can prevent human errors that can result when connection with the EMR system 30 is disrupted due to faulty updates to security software and other information technology (IT) outages, environmental disasters such as floods and fires, infrastructure disruptions such as communications and power outages, and even war. Thus, the medical device 100 can mitigate disruptions to clinical practices and opportunities for critical errors from delay of treatment to not having a complete medical history for a given patient.

The medical device 100 addresses the technical problem of data integrity and continuity failures in spot monitor systems when network connectivity to electronic medical record (EMR) systems is interrupted. Conventional spot monitors depend entirely on real-time network connections to central EMR systems for accessing patient data and recording physiological measurements. When these network connections fail due to system outages, infrastructure disruptions, or communication failures, conventional spot monitors lose access to critical patient information and cannot properly store newly captured physiological data, creating technical gaps in data persistence and patient monitoring capabilities.

This technical problem is particularly acute because spot monitors must operate continuously for patient safety, yet conventional implementations lack the technical architecture to maintain data integrity during network interruptions. The result is a technical failure where physiological measurements captured during outages are either lost entirely or stored in isolated systems that cannot be properly integrated with the patient's complete medical record.

The medical device 100 solves this technical problem through automated local caching architecture and intelligent data synchronization processes that are performed entirely by the spot monitor device without human intervention. This technical solution is enabled by the technical improvements in the memory management of the medical device 100 which include the EMR recovery module 206 that automatically retrieves and stores complete electronic medical records locally on the memory device 204 of the medical device 100. This technical implementation enables the medical device 100 to maintain full access to patient data even when network connectivity fails, representing a technical improvement over conventional spot monitors that become non-functional during outages.

Additionally, the technical solution provided by the medical device 100 is enabled by automated network monitoring capabilities that continuously detect connection status with the EMR system 30 and automatically switch operational modes when interruptions are detected. Upon detecting a network interruption, the medical device 100 automatically transitions from network-dependent operation to local EMR management mode, ensuring continuous data capture and storage without intervention from human operators, which represents a technical advancement in spot monitor reliability and autonomous operation.

During network interruptions, the medical device 100 automatically updates the locally cached EMR with physiological variable measurements captured by connected sensors, maintaining complete data integrity throughout the outage period. The medical device 100 performs these updates through automated processing operations that integrate sensor data directly into the local EMR structure, ensuring that no measurement data is lost and that the patient record remains current and complete. When network connectivity is restored, the medical device 100 automatically detects the reestablished connection and performs intelligent data synchronization, uploading the locally updated EMR to the central system. This automated synchronization process ensures data consistency between the local and central systems without requiring manual data entry or reconciliation processes.

A further technical improvement provided by the local storage of the EMR on the medical device 100 enables automated error checking for drug interactions and medication administration errors using the cached patient data, even during network outages. The medical device 100 can automatically scan medication barcodes, cross-reference against the patient's locally stored allergy and medication information, and issue automated alerts when potentially harmful interactions are detected. This technical capability maintains critical safety functions during system outages when conventional monitors would be unable to perform such checks.

Additionally, the medical device 100 includes automated memory management processes that determine when to purge cached EMR data based on configurable conditions such as time periods, patient discharge, or memory capacity limits. This automated data lifecycle management ensures optimal device performance while maintaining data security.

These technical improvements represent an advancement in spot monitor technology by transforming these devices from network-dependent peripherals into autonomous medical data management systems capable of maintaining full functionality during infrastructure failures. The disclosed technical architecture solves the technical problem of data loss and operational failure during network outages through specific technological implementations in device memory management, network monitoring, data synchronization, and automated processing capabilities. The medical device 100 improves the technology of spot monitors by providing technical resilience against network failures while maintaining complete data integrity and patient safety functions through automated, device-based operations.

The various embodiments described above are provided by way of illustration only and should not be construed to be limiting in any way. Various modifications can be made to the embodiments described above without departing from the true spirit and scope of the disclosure.

What is claimed is:

1. A medical device, comprising:
one or more modules configurable to connect to one or more sensors for capturing one or more physiological variable measurements;
a display screen for displaying the one or more physiological variable measurements;
at least one processing device; and
at least one memory device storing software instructions that, when executed by the at least one processing device, cause the at least one processing device to:
receive data identifying a patient;
retrieve an electronic medical record of the patient acquired from a system responsible for maintaining the electronic medical record;
store the electronic medical record on the at least one memory device;
determine an interruption of a connection with the system responsible for maintaining the electronic medical record; and
update the electronic medical record to include the physiological variable measurements captured by the one or more sensors while the interruption persists.

2. The medical device of claim 1, wherein the instructions, when executed by the at least one processing device, further cause the at least one processing device to:
determine whether one or more conditions are satisfied; and
purge the electronic medical record from the at least one memory device when the one or more conditions are satisfied.

3. The medical device of claim 2, wherein the one or more conditions include at least one of expiration of a predetermined time period and discharge of the patient.

4. The medical device of claim 1, wherein the instructions, when executed by the at least one processing device, further cause the at least one processing device to:
determine reestablishment of the connection with the system responsible for maintaining the electronic medical record; and
upload the electronic medical record including the one or more physiological variable measurements captured by the one or more sensors to the system responsible for maintaining the electronic medical record.

5. The medical device of claim 1, wherein store the electronic medical record on the at least one memory device is configurable to store one or more of physiological variable measurements, physical assessments, notes and history, diagnoses, test results, medication records, and diagnostic imaging obtained from other devices.

6. The medical device of claim 1, wherein the instructions, when executed by the at least one processing device, further cause the at least one processing device to:
display on the display screen diagnostic imaging stored in the electronic medical record of the patient, the diagnostic imaging captured by another device.

7. The medical device of claim 1, further comprising:
a microphone to capture audio from a user of the medical device; and
wherein the instructions, when executed by the at least one processing device, further cause the at least one processing device to:
process the audio to transcribe voice notes from the user of the medical device; and
store the voice notes to the electronic medical record.

8. The medical device of claim 1, wherein the instructions, when executed by the at least one processing device, further cause the at least one processing device to:
perform error checking for drug interactions and medication administration errors determined from the electronic medical record stored on the at least one memory device; and
issue an alarm when the error checking identifies a drug interaction potentially harmful to the patient or a medication administration error.

9. The medical device of claim 1, wherein the instructions, when executed by the at least one processing device, further cause the at least one processing device to:
display on the display screen reference publications relevant to a condition of the patient determined from the electronic medical record stored on the at least one memory device.

10. The medical device of claim 1, wherein the instructions, when executed by the at least one processing device, further cause the at least one processing device to:
provide a feature to toggle between a first screen displaying a monitoring of the physiological variable measurements captured by the one or more sensors and a second screen displaying the electronic medical record of the patient.

11. A method of monitoring a patient, the method comprising:
receiving data identifying a patient;
retrieving an electronic medical record of the patient acquired from a system responsible for maintaining the electronic medical record;
storing the electronic medical record on at least one memory device;
receiving one or more physiological variable measurements from one or more sensors;
determining an interruption of a connection with the system responsible for maintaining the electronic medical record; and
updating the electronic medical record to include the physiological variable measurements captured by the one or more sensors while the interruption persists.

12. The method of claim 11, further comprising:
determining whether one or more conditions are satisfied; and
purging the electronic medical record from the at least one memory device when the one or more conditions are satisfied.

13. The method of claim 12, wherein the one or more conditions include at least one of expiration of a predetermined time period, discharge of the patient, and reaching a capacity of the at least one memory device.

14. The method of claim 11, further comprising:
determining reestablishment of the connection with the system responsible for maintaining the electronic medical record; and
uploading the electronic medical record including the physiological variable measurements captured by the one or more sensors to the system responsible for maintaining the electronic medical record.

15. The method of claim 11, wherein store the electronic medical record on the at least one memory device is configurable to store one or more of physiological variable measurements, physical assessments, notes and history, diagnoses, test results, medication records, and diagnostic imaging obtained from other devices.

16. The method of claim 11, further comprising:
displaying on the display screen diagnostic imaging stored in the electronic medical record of the patient, the diagnostic imaging captured by another device.

17. The method of claim 11, further comprising:
processing audio to transcribe voice notes from a user of the medical device; and
storing the voice notes to the electronic medical record.

18. The method of claim 11, further comprising:
performing error checking for drug interactions and medication administration errors determined from the electronic medical record stored on the at least one memory device; and
issuing an alarm when the error checking identifies a drug interaction potentially harmful to the patient or a medication administration error.

19. The method of claim 11, further comprising:
displaying on the display screen reference publications relevant to a condition of the patient determined from the electronic medical record stored on the at least one memory device.

20. The method of claim 11, further comprising:
providing a feature to toggle between a first screen displaying a monitoring of the physiological variable measurements captured by the one or more sensors and a second screen displaying the electronic medical record of the patient.
